(19) United States
(12) Patent Application Publication
(10) Pub. No.: US 2005/0015211 A1
(43) Pub. Date: Jan. 20, 2005

(54) System, method, and program for estimating gene expression state, and recording medium therefor

(75) Inventors: Masataka Andoh, Tokyo (JP); Akira Saitoh, Tokyo (JP); Megu Ohtaki, Hatsukaichi-shi (JP); Kenichi Satoh, Hiroshima-shi (JP); Masahiko Nishiyama, Hiroshima-shi (JP); Keiko Otani, Tokyo (JP)

Correspondence Address: SUGHRUE MION, PLLC, 2100 PENNSYLVANIA AVENUE, N.W., SUITE 800, WASHINGTON, DC, 20037, US

(73) Assignees: NEC CORPORATION; Megu Ohtaki; JAPAN BIOLOGICAL IMFORMATICS CONSORTIUM

(21) Appl. No.: 10/766,011

(22) Filed: Jan. 29, 2004

(30) Foreign Application Priority Data
Jul. 17, 2003 (JP) 275983/2003

(51) Int. Cl. G06F 19/00; G01N 33/48; G01N 33/50

(52) U.S. Cl. 702/70

(57) ABSTRACT

A gene expression state estimating system according to the present invention includes an input device 1 for inputting microarray data, a program-controlled data analyzer 2, and an output device 3. The data analyzer 2 has parameter estimating means 21 and 22 for estimating distributed parameters for each component of a mixed normal distribution and a mixing ratio parameter using gene expression level data given from the input device 1, and posterior probability calculating means 23 for calculating the posterior probabilities of gene expression in each channel using each of the estimated parameters. The calculated posterior probabilities are outputted to the output device 3.

### BACKGROUND OF THE INVENTION

The present invention relates to a method and system for statistical analysis of cDNA microarray data using two different fluorescence dyes, and a recording medium for the same. In particular, the present invention relates to a system, method, and program for estimating the probability of gene expression in each channel, and a recording medium for the same.

Currently, the study of genomics is expanding from structural analysis on individual genes to systematic functional analysis of genes. Experiments using cDNA (complementary DNA) microarrays capable of simultaneously quantifying the expression levels of a large number of genes are expected to be extremely effective in functional analysis of functionally unknown genes or whole genes.

The objective of experiments using cDNA microarrays with two different fluorescence dyes is to detect the difference in gene expression level between two kinds of cells. The following gives a summary of a cDNA microarray configuration with two different fluorescence dyes. First, cDNAs of a large number of sets of genes are densely fixed on glass slides in arrays (microarrays) as reference probes.

Next, mRNAs extracted from two kinds of different conditional samples, cell 1 and cell 2 (e.g., normal cell and cancer cell), are labeled respectively with fluorescence dyes different in wavelength from each other to synthesize target cDNAs. Then, these cDANs are mixed in equal proportions, and hybridized with the microarrayed cDNAs or reference probes. After this competitive hybridization, the glass slides are imaged using a scanner and fluorescence intensities are measured separately for each dye. The fluorescence dye with which the cell 1 is labeled and the fluorescence dye with which the cell 2 is labeled are read from channel 1 and channel 2, respectively, to obtain gene expression level data (microarray data).

Thus, since the process of obtaining microarray data is so complicated as to require advanced experimental techniques, it is conceivable that several experimental errors could occur at each stage of the experiment. Therefore, in order to retrieve data to be truly biologically significant from the microarray data, analyzing expression level distributions and experimental errors presents a significant challenge to be solved.

In regard to the expression level distributions, prior art document 1 (Newton et. al., 2001, Journal of Computational Biology, Vol. 8, pp. 37-52) can be referred to, for example, in which Newton et. al. forms a hypothesis that proposes the use of the gamma distribution function to help analyze expression levels so as to consider statistical characteristics about the ratio of expression levels (the ratio of expression levels in channel 1 and channel 2).
f(x)=pφ(x−μ₁|σ₁²)+(1−p)φ(x−μ₂|σ₂²) (1)

In regard to the observed expression level data, prior art document 2 (Lee et. al., 2000, Proceeding of the National Academy of Sciences, Vol. 97, No. 18, pp. 9834-9839) can be referred to, for example. Assuming the ability to separate true expression levels into two levels and the existence of accidental errors, Lee et. al. adopts a mixed normal distribution as shown in the following equation (1) to consider statistical characteristics about the expression level data:

Here, x denotes (the logarithmic value of) a gene expression level such as fluorescence intensity obtained with a scanner or the like. Further, the first term of the right hand side, φ(x−μ₁|σ₁²), represents a normal distribution with average μ₁ and variance σ₁² when a gene is being expressed, the second term φ(x−μ₂|σ₂²) represents the density function of a normal distribution with average μ₂ and variance σ₂² when no gene is being expressed, and p is a parameter representing the mixing ratio.

In regard to the analysis of the experimental errors, there have been proposed several methods of removing systematic errors, so-called normalization methods. For example, when referring to prior art document 3 (Chen et. al., 1997, Journal of Biomedical Optics, Vol. 2, pp. 364-374), Chen et. al. assumes that the median values of gene expression levels of two cells are equal to correct for the measured values obtained from channel 1 and channel 2, respectively. Further, when referring to prior art document 4 (Dudoit et. al., 2000, “Statistical methods for identifying differentially expressed genes in replicated cDNA microarray experiments,” Technical Report #5782), prior art document 5 (Schuchhardt et. al., 2000, Nucleic Acids Research, Vol.28, No. 10), and prior art document 6 (Yang et. al., 2002, Nucleic Acids Research, Vol.30, No.4), Dudoit, Schuchhardt, and Yang consider that systematic errors are caused by different locations of spots on glass slides or different sensitivities of the two kinds of fluorescence dyes, and propose methods of removing the errors.

The above-mentioned prior art problems are derived from the fact that the analytical results of microarray data lack reproducibility because of low precision and efficiency. It is considered that the cause is insufficient separation of microarray data into true signals, and systematic and measurement errors in the conventional analytical methods. Therefore, removal of systematic errors and evaluation of measurement errors are important issues.

In regard to the removal of systematic errors, a copending patent application entitled “Method and System for Correction of cDNA Microarray Data, and Recoding Medium Therefor” has been filed separately. Therefore, the present invention assumes that systematic errors are already removed from the microarray data.

The conventional analytical methods using microarray data deal with only the ratio (the difference of logarithmic values) of gene expression levels of two channels, that is, they do not deal with the gene expression level of each channel, for the reason that quantitative uniformity of cDNA in each spot is not ensured. Therefore, the conventional analytical methods results in insufficient separation between true signals related to gene expression state and measurement errors.

### SUMMARY OF THE INVENTION

It is an object of the present invention to provide a method and system for separating true signals related to gene expression from measurement errors to increase the precision and efficiency of analysis using microarray data, and further estimating the probability of gene expression in each channel.

A gene expression state estimating system of the present invention includes an input device for inputting microarray data, a program-controlled data analyzer, and an output device. The data analyzer has parameter estimating means for estimating distributed parameters for each component of a mixed normal distribution and a mixing ratio parameter using gene expression level data given from the input device, and posterior probability calculating means for calculating the posterior probabilities of gene expression in each channel using each of the estimated parameters. The calculated posterior probabilities are outputted to the output device.

The adoption of such a configuration to estimate the state of gene expression can attain the object of the present invention.

### BRIEF DESCRIPTION OF THE DRAWINGS

FIG. 1 is a schematic graph of a mathematical model using S-D plots according to the present invention.

FIG. 2 is a block diagram showing the structure of a first embodiment according to the present invention.

FIG. 3 is a flowchart showing the operation of the first embodiment according to the present invention.

FIG. 4 is a block diagram showing the structure of a second embodiment according to the present invention.

FIG. 5 is a cumulative distribution graph showing an estimated normal distribution of gene expression level data near V=0.

FIG. 6 is a graph showing the density function of the estimated normal distribution of the gene expression level data near V=0.

FIG. 7 is a graph showing S-D plots of gene expression level data.

### DESCRIPTION OF THE PREFERRED EMBODIMENTS

A mathematical model of gene expression level data obtained from a microarray according to the present invention will first be described. If X denotes the gene expression level of cell 1 obtained with channel 1 and Y denotes the gene expression level of cell 2 obtained with channel 2, then respective gene expression level data are shown in the following equation (2)
X=τ₁α+β+ε₁
Y=τ₂α+β+ε₂ (2)
where X and Y denote amounts subjected to adequate transformations of observed values including logarithmic transformation or power transformation and linear transformation.

Here, τ1 and τ2 take either 1 or 0, which represents the presence or absence (ON/OFF) of true gene expression in each cell. Further, α denotes the amount of mRNA produced when the gene is ON-state and a random variable of gene expression defined by the state of the spot, β denotes a common measurement error between the channel 1 and the channel 2, and ε denotes a measurement error independent between the channels. Note that each distribution of random variables follows the following equation (3)

$$\begin{matrix}log α ∼ N ( μ - \frac{{λ}^{2}}{2} , {λ}^{2} ) \\ {ɛ}_{j} ∼ N ( 0 , {σ}_{ɛ}^{2} ) , j = 1 , 2 \\ β ∼ N ( 0 , {σ}_{β}^{2} )\end{matrix} \qquad ( 3 )$$

Here, N (μ, σ²) demotes a one-dimensional normal distribution with average μ and variance σ². Further, α, β, and ε are all independent. In this mathematical model, when a gene is being expressed (ON-state), the true expression level is a random variable that takes on nonnegative values, while when it is not being expressed (OFF-state), only simple measurement errors are considered to be observed. Further, referring to the prior art document 6, an S-D transformation is performed as a modification from the M-A transformation adopted by Yang Y. H. et. al. as shown in the following equation (4):
U=X+Y,
V=X−Y (4)

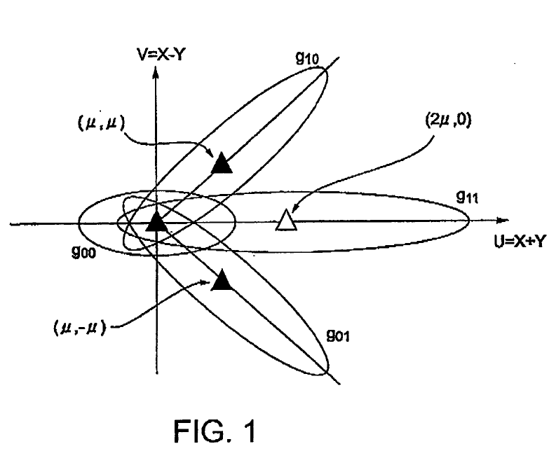

In other words, the transformation is made assuming that U and V are the sum and difference of gene expression levels of two channels, respectively. A schematic graph of this S-D transformation model is shown in FIG. 1. Note that this plot is called the S-D plot. In FIG. 1, goo represents a simultaneous distribution when no gene is being expressed in both cells, g₁₀ represents a simultaneous distribution when a gene is being expressed in cell 1 but not in cell 2, g₀₁ represents a simultaneous distribution when a gene is being expressed in cell 2 but not in cell 1, and g₁₁ represents a simultaneous distribution when any gene are being expressed in both cells. The density function of the distribution g₀₀ is shown in the following equation (5)
g₀₀(u,ν|θ)=φ(u|4σ_{β}²+2σ_{ε}²)φ(ν|2σ_{ε}²) (5)

Here, φ(u|σ²) is the density function of a one-dimensional normal distribution with average 0 and variance σ². The density function of the distribution g₁₀ is shown in the following equation (6):

$$\begin{matrix}{g}_{10} ( u , v \vert θ ) = {\int }_{- ∞}^{+ ∞} φ ( u - μ {ⅇ}^{- \frac{{λ}^{2}}{2} + λ z} \vert 4 {σ}_{β}^{2} + 2 {σ}_{ɛ}^{2} ) φ \\ ( v - μ {ⅇ}^{- \frac{{λ}^{2}}{2} + λ z} \vert 2 σ \frac{2}{ɛ} ) φ ( z \vert 1 ) ⅆ z \\ ≅ {φ}_{2} ( u - μ , v - μ \vert {\sum }_{10} )\end{matrix} \qquad ( 6 )$$

Here, φ²(u, v|Σ) is the density function of a two-dimensional normal distribution with average vector 0 and variance-covariance matrix Σ, and Σ₁₀ is a 2×2 variance-covariance matrix, which is shown in the following equation (7)

$${\sum }_{10} = ( \begin{matrix}{μ}^{2} ( {ⅇ}^{{λ}^{2}} - 1 ) + 4 {σ}_{β}^{2} + 2 {σ}_{ɛ}^{2} & {μ}^{2} ( {ⅇ}^{{λ}^{2}} - 1 ) \\ {μ}^{2} ( {ⅇ}^{{λ}^{2}} - 1 ) & {μ}^{2} ( {ⅇ}^{{λ}^{2}} - 1 ) + 2 {σ}_{ɛ}^{2}\end{matrix} ) \qquad ( 7 )$$

The density function of the distribution g₀₁ is shown in the following equation (8):

$$\begin{matrix}{g}_{01} ( u , v \vert θ ) = {\int }_{- ∞}^{+ ∞} φ ( u - μ {ⅇ}^{- \frac{{λ}^{2}}{2} + λ z} \vert 4 {σ}_{β}^{2} + 2 {σ}_{ɛ}^{2} ) φ & ( 8 ) \\ ( v - μ {ⅇ}^{- \frac{{λ}^{2}}{2} + λ z} \vert 2 σ \frac{2}{ɛ} ) φ ( z \vert 1 ) ⅆ z & \\ ≅ {φ}_{2} ( u - μ , v - μ \vert {\sum }_{01} ) & ( 8 )\end{matrix}$$

Here, Σ₀₁ is a 2×2 variance-covariance matrix, which is shown in the following equation (9):

$${\sum }_{01} = ( \begin{matrix}{μ}^{2} ( {ⅇ}^{{λ}^{2}} - 1 ) + 4 {σ}_{β}^{2} + 2 {σ}_{ɛ}^{2} & - {μ}^{2} ( {ⅇ}^{{λ}^{2}} - 1 ) \\ - {μ}^{2} ( {ⅇ}^{{λ}^{2}} - 1 ) & {μ}^{2} ( {ⅇ}^{{λ}^{2}} - 1 ) + 2 {σ}_{ɛ}^{2}\end{matrix} ) \qquad ( 9 )$$

The density function of the distribution g₁₁ is shown in the following equation (10)

$$\begin{matrix}{g}_{11} ( u , v \vert θ ) = φ ( v \vert 2 {σ}_{ɛ}^{2} ) {\int }_{- ∞}^{+ ∞} φ ( u - μ {ⅇ}^{- \frac{{λ}^{2}}{2} + λ z} \vert 4 {σ}_{β}^{2} + 2 {σ}_{ɛ}^{2} ) φ \\ ( z \vert 1 ) ⅆ z \\ ≅ φ ( u - 2 μ \vert 4 {μ}^{2} ( {ⅇ}^{{λ}^{2}} - 1 ) + 4 {σ}_{β}^{2} + 2 {σ}_{ɛ}^{2} ) φ ( v \vert 2 {σ}_{ɛ}^{2} )\end{matrix} \qquad ( 10 )$$

Based on the above-mentioned distributions, posterior probabilities of gene expression in cell 1 and cell 2 are shown in the following equations (11) and (12)

$$\begin{matrix}Pr ( {τ}_{1} = 1 \vert p , θ ) = \frac{{p}_{10} {g}_{10} ( u , v \vert θ ) + {p}_{11} {g}_{11} ( u , v \vert θ )}{f ( u , v \vert p , θ )} & ( 11 ) \\ Pr ( {τ}_{2} = 1 \vert p , θ ) = \frac{{p}_{01} {g}_{01} ( u , v \vert θ ) + {p}_{11} {g}_{11} ( u , v \vert θ )}{f ( u , v \vert p , θ )} & ( 12 )\end{matrix}$$

where f(u, v|p, θ) is given by the following equation (13)

$$f ( u , v \vert p , θ ) = {\sum }_{( j , k ) ∈ {\{ 0 , 1 \}}^{2}}^{} {p}_{jk} {g}_{jk} ( u , v \vert θ ) \qquad ( 13 )$$

Note that p=(p₀₀, p₁₀, p₀₁, p₁₁) is a parameter representing the mixing ratio for each distribution.

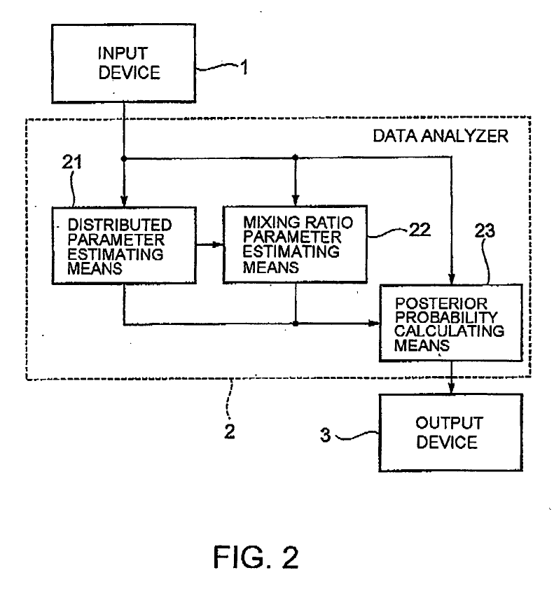

An embodiment of the present invention will next be described in detail with reference to the accompanying drawings. Referring to FIG. 2, the first embodiment of the present invention is a system for estimating posterior probabilities of gene expression states in cell 1 and cell 2 through the process of formulating a mathematical model related to gene expression level data and the process of estimating unknown parameters by the application of the formulated mathematical model to the data analysis, and using the calculated estimates of parameters. The system includes an input device 1 such as a keyboard, a program-controlled data analyzer 2, and an output device 3 such as a display device or printer.

The data analyzer 2 is provided with distributed parameter estimating means 21, mixing ratio parameter estimating means 22, and posterior probability calculating means 23. The distributed parameter estimating means 21 estimates distributed parameters for each component in a mixed normal distribution using gene expression level data from the input device 1. The estimated distributed parameters are sent to the mixing ratio parameter estimating means 22 and the posterior probability calculating means 23. The mixing ratio parameter estimating means 22 estimates a mixing ratio parameter for the mixed normal distribution by a conditional maximum likelihood method using the gene expression level data from the input device 1 and the distributed parameters for each component given from the distributed parameter estimating means 21. The estimated mixing ratio parameter is sent to the posterior probability calculating means 23. The posterior probability calculating means 23 calculates the posterior probability of a gene expression state in each channel using the gene expression level data from the input device 1, the distributed parameters for each component given from the distributed parameter estimating means 21, and the mixing ratio parameter from the mixing ratio parameter estimating means 22. The calculated posterior probability is sent to the output device 3.

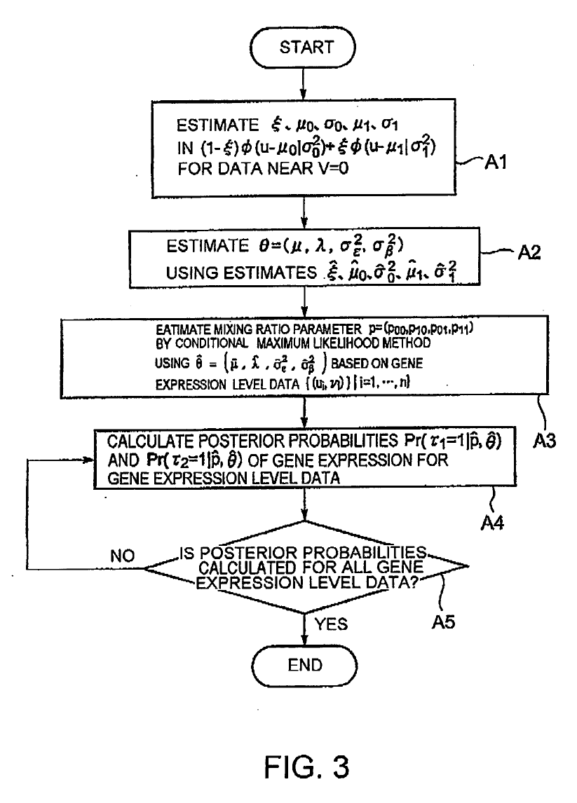

Referring next to FIGS. 2 and 3, the process of formulating a mathematical model related to gene expression level data and the process of estimating unknown parameters by the application of the formulated mathematical model to the data analysis will be described in detail. Gene expression level data {(uᵢ, νᵢ)|i=1, . . . , n} given from the input device 1 is sent to the distributed parameter estimating means 21 and the mixing ratio parameter estimating means 22. The distributed parameter estimating means 21 estimates ξ, μ₀, σ₀, μ₁, and σ₁ by applying the mixed normal distribution of two components, as shown in the following equation (14), to data {uᵢ||νᵢ≦c_{M}, i=1, . . . , n} on the sum of the amounts of expression of genes near V=0 where c_{M} denotes the median value of the absolute difference |νᵢ|(i=1, . . . ,n) of gene expression levels (step A1 in FIG. 3)
(1−ξ)φ(u−μ₀|σ₀²)+ξφ(u−μ₁|σ₁²) (14)
where φ(*|σ²) is the density function of a one-dimensional normal distribution with average 0 and variance σ², (μ₀, σ₀²) and (μ₁, σ₁²) are average and variance parameters for first and second components, respectively, and ξ is the mixing ratio, with the assumption that μ₀<μ₁, σ₀²>0, σ₁²>0, 0<ξ<1 is satisfied.

Next, the distributed parameter estimating means 21 uses the estimated \hat{ξ}, \hat{μ}₀, \hat{σ}₀², \hat{μ}₁, \hat{σ}₁² to estimate μ, σ_{ε}², σ_{β}², λ according to the following equations (15), (16), (17), and (18) (step A2)

$$\begin{matrix}\hat{μ} = ( {\hat{μ}}_{1} - {\hat{μ}}_{0} ) / 2 & ( 15 ) \\ {\hat{σ}}_{ɛ}^{2} = \frac{1}{2 ‖ {N}_{0} ‖} {\sum }_{i ∈ {N}_{0}}^{} {v}_{i}^{2} & ( 16 ) \\ {\hat{σ}}_{β}^{2} = \frac{1}{4} {\hat{σ}}_{0}^{2} - \frac{1}{2} {\hat{σ}}_{ɛ}^{2} & ( 17 ) \\ \hat{λ} = \sqrt{log ( 1 + \frac{{\hat{σ}}_{1}^{2} - {\hat{σ}}_{2}^{2}}{4 {\hat{μ}}^{2}} )} & ( 18 )\end{matrix}$$

where N₀ denotes an index set of data values that satisfies i ε {i|uᵢ<\hat{μ}₀} and ∥N₀∥ denotes the number of elements.

Next, the mixing ratio parameter estimating means 22 estimates a mixing ratio parameter p=(p₀₀, p₁₀, p₀₁, p₁₁) by a conditional maximum likelihood method using an estimate \hat{θ}=(\hat{μ}, \hat{λ}, \hat{σ}_{ε}², \hat{σ}_{β}²) of each parameter given from the distributed parameter estimating means 21 by applying a two-variable mixed normal distribution of four components shown in the following equation (19) to the gene expression level data {(uᵢ, vᵢ)|i=1, . . . ,n} given from the input device 1 (step A3).
p₀₀g₀₀(u,ν|\hat{θ})+p₁₀g₁₀(u,ν|\hat{θ})+p₀₁g₀₁(u,ν|\hat{θ})+p₁₁g₁₁(u,ν|\hat{θ})=p₀₀φ(u|4\hat{σ}_{β}²+2\hat{σ}_{ε}²)φ(ν|2\hat{σ}_{ε}²)+p₁₀φ₂(u−\hat{μ},ν−\hat{μ}|Σ₁₀)+p₀₁φ₂(u−\hat{μ},ν+\hat{μ}|Σ₀₁)+p₁₁φ(u−2\hat{μ}|4\hat{μ}²(e^{λ}^{²}−1)+4\hat{σ}_{β}²+2\hat{σ}_{ε}²)φ(ν|2\hat{σ}_{ε}²) (19)

Here, it is assumed that the above equation satisfies the relationships shown in the following equation (20) (where \hat{Σ}₁₀ is a 2×2 variance-covariance matrix derived from the equation (7)) and the following equation (21) (where \hat{Σ}₀₁ is a 2×2 variance-covariance matrix derived from the equation (9)).

$$\begin{matrix}{\hat{\sum }}_{10} = ( \begin{matrix}{\hat{μ}}^{2} ( {ⅇ}^{{\hat{λ}}^{2}} - 1 ) + 4 {\hat{σ}}_{β}^{2} + 2 {\hat{σ}}_{ɛ}^{2} & {\hat{μ}}^{2} ( {ⅇ}^{{\hat{λ}}^{2}} - 1 ) \\ {\hat{μ}}^{2} ( {ⅇ}^{{\hat{λ}}^{2}} - 1 ) & {\hat{μ}}^{2} ( {ⅇ}^{{\hat{λ}}^{2}} - 1 ) + 2 {\hat{σ}}_{ɛ}^{2}\end{matrix} ) & ( 20 ) \\ {\hat{\sum }}_{01} = ( \begin{matrix}{\hat{μ}}^{2} ( {ⅇ}^{{\hat{λ}}^{2}} - 1 ) + 4 {\hat{σ}}_{β}^{2} + 2 {\hat{σ}}_{ɛ}^{2} & - {\hat{μ}}^{2} ( {ⅇ}^{{\hat{λ}}^{2}} - 1 ) \\ - {\hat{μ}}^{2} ( {ⅇ}^{{\hat{λ}}^{2}} - 1 ) & {\hat{μ}}^{2} ( {ⅇ}^{{\hat{λ}}^{2}} - 1 ) + 2 {\hat{σ}}_{ɛ}^{2}\end{matrix} ) & ( 21 )\end{matrix}$$

The process of estimating posterior probabilities of gene expression states in cell 1 and cell 2 using the calculated estimates of parameters will next be described.

The posterior probability calculating means 23 can describe the posterior probabilities of gene expression state in each cell for each pair (u, v) of the gene expression level data given from the input device 1 using the estimates \hat{θ}=(\hat{μ}, \hat{λ}, \hat{σ}_{ε}², \hat{σ}_{β}²) and \hat{p}=(\hat{p}₀₀, \hat{p}₁₀, \hat{p}₁₁) of each parameter given from the distributed parameter estimating means 21 and the mixing ratio parameter estimating means 22.

In other words, the posterior probabilities indicating that any gene expression is ON-state in cell 1 and cell 2 can be calculated from the following equations (22) and (23) (step A4).

$$\begin{matrix}Pr ( {τ}_{1} = 1 \vert \hat{p} , \hat{θ} ) = \frac{{\hat{p}}_{10} {g}_{10} ( u , v \vert \hat{θ} ) + {\hat{p}}_{11} {g}_{11} ( u , v \vert \hat{θ} )}{f ( u , v \vert \hat{p} , \hat{θ} )} & ( 22 ) \\ Pr ( {τ}_{2} = 1 \vert \hat{p} , \hat{θ} ) = \frac{{\hat{p}}_{01} {g}_{01} ( u , v \vert \hat{θ} ) + {\hat{p}}_{11} {g}_{11} ( u , v \vert \hat{θ} )}{f ( u , v \vert \hat{p} , \hat{θ} )} & ( 23 )\end{matrix}$$

It is then judged whether calculations of posterior probabilities indicating that any gene expression is ON-state have been made for all the pairs (u, v) of the gene expression level data (step A5). When all the calculations have been completed, the process is ended, while when all the calculations have not been completed yet, the posterior probability related to the next gene is calculated.

The calculated posterior probabilities of gene expression in each channel are sent to the output device 3. The output device 3 displays or prints out the posterior probabilities of gene expression in each channel in the form of a graph.

The following describes the effects of the embodiment. In the embodiment, a mathematical model in which the concept of gene expression/nonexpression is introduced is constructed to separate true signals from experimental errors. Further, the use of data on the sum and difference of gene expression levels in two channels makes it easy to obtain information on the sensitivity of microarray data to fluorescence intensities in each channel, allowing more accurate extraction of the magnitude of experimental errors. Furthermore, a two-dimensional simultaneous distribution is described for these sum and difference data. It allows high-precision estimation of posterior probabilities of gene expression in each channel.

In addition, the posterior probability indicating an event of differential expression between cell 1 and cell 2 (mismatched ON-OFF state) (step 4 in FIG. 3) is calculated by the following equation (24).,

$$Pr ( {τ}_{1} \neq {τ}_{2} \vert \hat{p} , \hat{θ} ) = \frac{{\hat{p}}_{10} {g}_{10} ( u , v \vert \hat{θ} ) + {\hat{p}}_{01} {g}_{01} ( u , v \vert \hat{θ} )}{f ( u , v \vert \hat{p} , \hat{θ} )} \qquad ( 24 )$$

Thus, the embodiment has the advantage of detecting candidate genes that are likely to reveal differential expression in cell 1 and cell 2.

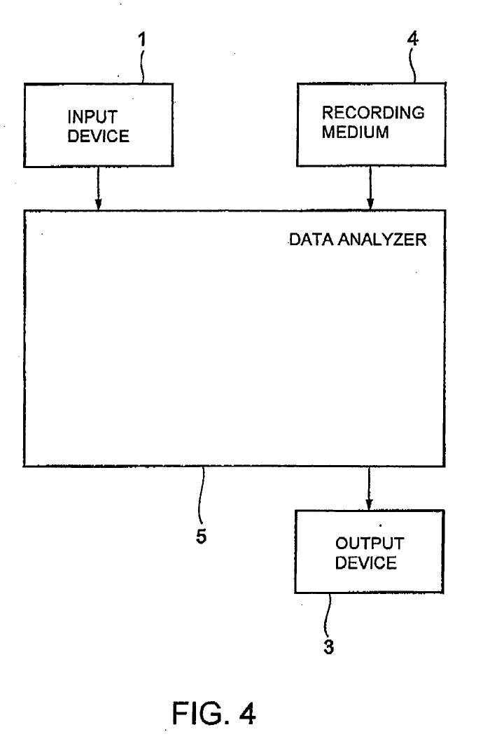

A second embodiment of the present invention will next be described in detail with reference to FIG. 4. Like the first embodiment, the second embodiment of the present invention includes the input device, data analyzer, and the output device. The second embodiment also includes a recording medium 4 with a data analyzing program recorded on it. The recording medium 4 may be either portable or fixed type, such as a magnetic disk, semiconductor memory, CD-ROM, or any other recording medium. Alternatively, a computer program capable of executing the method of the present invention may be stored in a memory device of a computer connected to a network so that it can be transferred to another computer through the network. The form of the medium that provides a computer program executing the algorithm is a distributable as a medium readable in a variety of computer formats, and is not limited to a specific type.

The data analyzing program is read from the recording medium 4 into a data analyzer 5 to control the operation of the data analyzer 5 to execute processing on data files inputted from the input device 1 in the same manner as the data analyzer 2 does in the first embodiment.

The following specifically describes the embodiments of the present invention. Data used as an example is obtained from an experiment for comparing the states of gene expression of two different types of cancer cells (cell 1 and cell 2).

The test is conducted on expression patterns of 48 grids on one chip, 441 (21×21) spots pergrid, a total of 21168 genes.

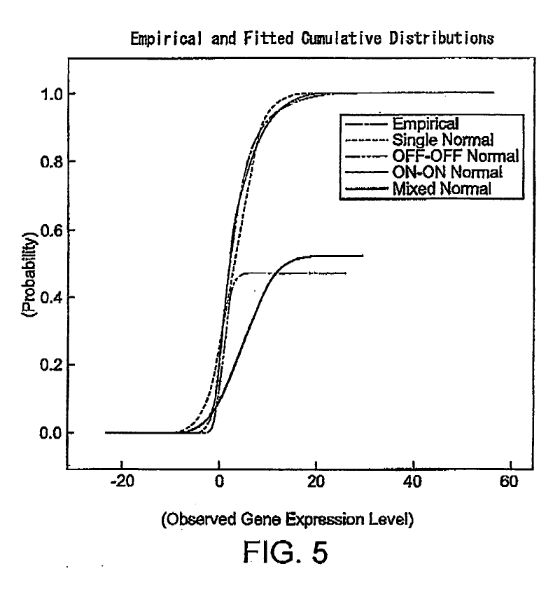

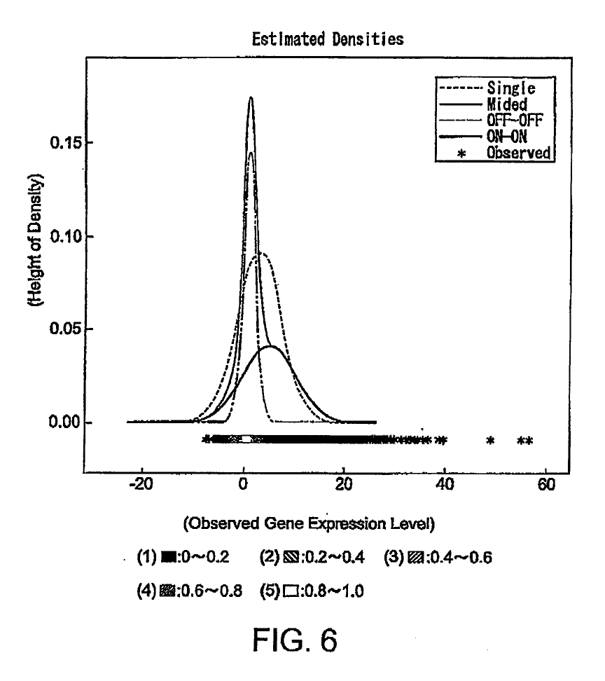

FIGS. 5 and 6 show estimation results of each of the distributions U of the sum of expression levels when cell 1 and cell 2 both show OFF or ON state of gene expression (V=0), a mixed normal distribution, and a single-peaked normal distribution for contrast purposes. The following table 1 shows estimation results of distributed parameters for each component (results of step A1).

TABLE 1
Result of N2MIXFit (Ver 0.998)

| | |
|---|---|
| Name of Data Set to be analyzed = | n145h1.std |
| Name of Target Variate = | S_CH1 |
| Type of Transformation = | 1/16 |
| Sample size = | 14726 |
| Critical Value for Convergence = | .10000E−06 |
| Iterations for Convergence = | 50 |
| Job Termination Status = | Normally Terminated |

| | Mean | SD | Rate(%) |
|---|---|---|---|
| Single Component: | 3.0273 | 4.8560 | 100.00 |
| 1st Component: | .89956 | 1.2978 | 47.36 |
| 2nd Component: | 4.9490 | 5.1395 | 52.62 |

| | |
|---|---|
| Log_Likelihood for Single Component = | −42565. |
| for Two Components Mixed = | −40465. |
| Log of Likelihood Ratio Statistics = | 2099.8 |

FIG. 5 shows cumulative distribution functions and FIG. 6 shows density functions, in which the thin solid line indicates the estimation results of an assumed mixed normal distribution, the chain double-dashed line indicates the estimation results of its first component (OFF-OFF), the bold solid line indicates the estimation results of its second component (ON-ON), and the dashed line indicates the estimation results of an assumed single-peaked normal distribution.

The long and short dashed line in FIG. 5 indicates an empirical cumulative distribution function based on observed data, showing that it well follows the curve of the mixed normal distribution (thin solid line) in which observed values are estimated.

In FIG. 6, the asterisk marks (which ar e replaced with the following hatching patterns (1) to (5) in the range of gene expression levels from about 0 to 30 because, though the asterisk marks can be discernible at both ends, they are densely overlapped within the range) indicates observed data. The hatching patterns ((1) to (5)) represent the magnitude of posterior probability values belonging to the first component. In other words, the solidly shaded area (1) indicates the range of posterior probabilities from 0 to 0.2, the hatching area (2) from 0.2 to 0.4, the hatching area (3) from 0.4 to 0.6, the hatching area (4) from 0.6 to 0.8, and the hatching area (5) from 0.8 to 1.0.

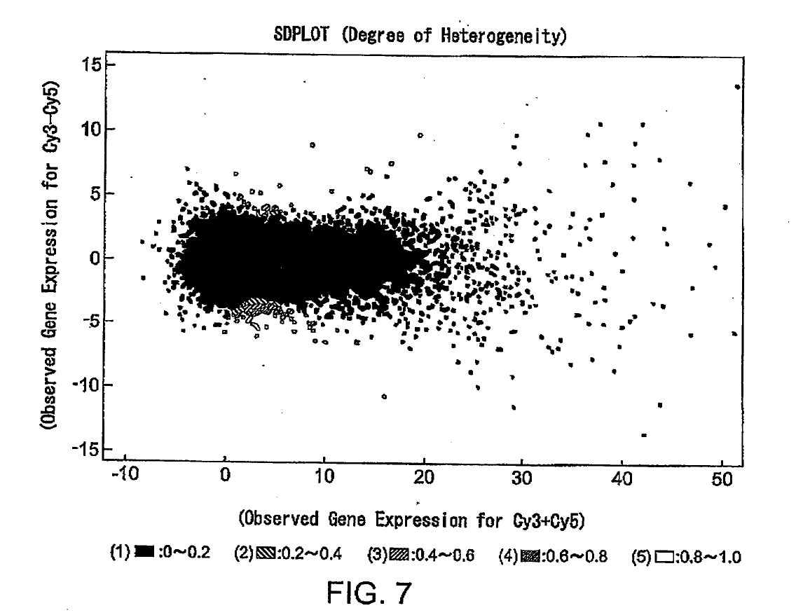

FIG. 7 shows an S-D plot of gene expression level data, in which the abscissa indicates the sum of logarithmic values of gene expression levels of cell 1 and cell 2, and the ordinate indicates the different between the logarithmic values. In FIG. 7, the hatching patterns represent the magnitude of posterior probabilities indicating mismatched expression states (ON-OFF or OFF-ON) between cell 1 and cell 2. In other words, the solidly shaded area (1) indicates the range of posterior probabilities from 0 to 0.2, the hatching area (2) from 0.2 to 0.4, the hatching area (3) from 0.4 to 0.6, the hatching area (4) from 0.6 to 0.8, and the hatching area (5) from 0.8 to 1.0.

The following table (2) shows estimation results of distributed parameters by the conditional maximum likelihood method (results of steps A2 and A3 in FIG. 3). A gene corresponds to each plotted spot in FIG. 7, and this makes it easy to narrow down gene candidates related to the difference between cell 1 and cell 2.

TABLE 2
RESULT OF MAD Ver(0.998)

Transformation = 6 (1/16)
Sample Size = 21168

| | |
|---|---|
| SIGMA_Epsilon = | .898 |
| MU = | 2.023 |
| LAMBDA = | .060 |
| SIGMA_Beta = | .138 |
| P11 = | .561 |
| P10 = | .004 |
| P01 = | .011 |
| P00 = | .425 |

The first effect of the present invention is to achieve a separation between true signals related to gene expression and experimental errors by introducing the concept of gene expression/nonexpression into the gene expression level data obtained from microarrays to construct a mathematical model.

The second effect of the present invention is to make it easy to obtain sensitivity information on the sensitivity of microarray data to fluorescence intensities of two channels by transforming the gene expression level data obtained from microarrays into data on the sum and difference of the gene expression levels between two channels. It then makes it possible to visualize the magnitude of experimental errors.

The third effect of the present invention is to enable estimation of posterior probability related to expression/nonexpression of each gene in each of the two channels by transforming the gene expression level data obtained from microarrays into data on the sum and difference of the gene expression levels between two channels to describe a two-dimensional simultaneous distribution of the sum and difference data. It then makes it possible to detect genes related to differences between cell 1 and cell 2 with high precision.

1. A gene expression state estimating system for estimating the probability of gene expression in each channel, the system including an input device for sending gene expression level data, a program-controlled data analyzer, and an output device, wherein said data analyzer comprises distributed parameter estimating means for estimating distributed parameters of a mixed normal distribution shown in the following equation (25) using the gene expression level data from said input device, and sending the estimated distributed parameters:
(1−ξ)φ(u−μ₀|σ₀²)+ξφ(u−μ₁|σ₁²) (25)
where φ(*|σ²) represents the density function of a one-dimensional normal distribution with average 0 and variance σ², (μ₀, σ₀²) and (μ₀, σ₁²) are average and variance parameters of first and second components, respectively, and t is the mixing ratio, with the assumption that μ₀<μ₁, σ₀²>0, σ₁²>0, 0<ξ<1 is satisfied,
mixing ratio parameter estimating means for estimating a mixing ratio parameter of the mixed normal distribution using the gene expression level data sent from said input device and the distributed parameters sent from said distributed parameter estimating means, and sending the estimated mixing ratio parameter, and
posterior probability calculating means for calculating the posterior probability of the expression state of each gene in each channel using the gene expression level data, the estimated distributed parameters and mixing ratio parameter, and sending the calculated posterior probability to said output device.

2. The system according to claim 1 wherein said distributed parameter estimating means estimates the mixing ratio (ξ), average (μ₀, μ₁), and variance (σ₀², σ₁²) by applying the mixed normal distribution of two components to data on the sum of the amounts of expression of genes located in a region where the difference of gene expression levels X and Y of two channels is near 0.

3. The system according to claim 2 wherein when the median value of the absolute difference |νᵢ|(i=1, . . . , n) of the gene expression levels X and Y is CM, the data on the amounts of gene expression is shown by {uᵢ∥νᵢ|≦c_{M}, i=1, . . . ,n}.

4. The system according to claim 3 wherein said distributed parameter estimating means performs estimation by the use of the estimated \hat{ξ}, \hat{μ}₀, \hat{σ}₀², \hat{μ}₁, \hat{σ}₁² according to the following equations (26), (27), (28), and (29) to estimate a, as, μ, σ_{ε}², σ_{β}², λ;
$$\begin{matrix}\hat{μ} = ( {\hat{μ}}_{1} - {\hat{μ}}_{0} ) / 2 & ( 26 ) \\ {\hat{σ}}_{ɛ}^{2} = \frac{1}{2 ‖ {N}_{0} ‖} {\sum }_{i ∈ {N}_{0}} {v}_{i}^{2} & ( 27 ) \\ {\hat{σ}}_{β}^{2} = \frac{1}{4} {\hat{σ}}_{0}^{2} - \frac{1}{2} {\hat{σ}}_{ɛ}^{2} & ( 28 ) \\ \hat{λ} = \sqrt{log ( 1 + \frac{{\hat{σ}}_{1}^{2} - {\hat{σ}}_{2}^{2}}{4 {\hat{μ}}^{2}} )} & ( 29 )\end{matrix}$$
where N₀ denotes an index set of data values that satisfies i ε {i|uᵢ<\hat{μ}₀} and ∥N₀∥ denotes the number of elements.

5. The system according to claim 4 wherein said mixing ratio parameter estimating means estimates the mixing ratio parameter p=(p₀₀, p₁₀, p₀₁, p₁₁) (where p₀₀ denotes a mixing ratio when no gene is being expressed in both cells 1 and 2, p₁₁ denotes a mixing ratio when any gene is being expressed in both cells 1 and 2, p₁₀ denotes a mixing ratio when a gene is being expressed in cell 1 but not in cell 2, and g₀₁ represents a mixing ratio when a gene is being expressed in cell 2 but not in cell 1) using \hat{θ}=(\hat{μ}, \hat{λ}, \hat{σ}_{ε}², \hat{σ}_{β}²) given from said distributed parameter estimating means by applying a two-variable mixed normal distribution of four components shown in the following equation (30) to the gene expression level data {(uᵢ, vᵢ)i=1, . . . , n} sent from said input device
p₀₀g₀₀(u,ν|\hat{θ})+p₁₀g₁₀(u,ν|\hat{θ})+p₀₁g₀₁(u,ν|\hat{θ})+p₁₁g₁₁(u,ν|\hat{θ})=p₀₀φ(u|4\hat{σ}_{β}²+2\hat{σ}_{ε}²)φ(ν|2\hat{σ}_{ε}²)+p₁₀φ₂(u−\hat{μ},ν−\hat{μ}|Σ₁₀)+p₀₁φ₂(u−\hat{μ},ν+\hat{μ}|Σ₀₁)+p₁₁φ(u−2\hat{μ}|4\hat{μ}²(e^{λ}^{²}−1)+4\hat{σ}_{β}²+2\hat{σ}_{ε}²)φ(ν|2\hat{σ}_{ε}²) (30)
where the above equation satisfies the relationships shown in the following equations (31) and (32).
$$\begin{matrix}{\hat{\sum }}_{10} = ( \begin{matrix}{\hat{μ}}^{2} ( {ⅇ}^{{\hat{λ}}^{2}} - 1 ) + 4 {\hat{σ}}_{β}^{2} + 2 {\hat{σ}}_{ɛ}^{2} & {\hat{μ}}^{2} ( {ⅇ}^{{\hat{λ}}^{2}} - 1 ) \\ {\hat{μ}}^{2} ( {ⅇ}^{{\hat{λ}}^{2}} - 1 ) & {\hat{μ}}^{2} ( {ⅇ}^{{\hat{λ}}^{2}} - 1 ) + 2 {\hat{σ}}_{ɛ}^{2}\end{matrix} ) & ( 31 ) \\ {\hat{\sum }}_{01} = ( \begin{matrix}{\hat{μ}}^{2} ( {ⅇ}^{{\hat{λ}}^{2}} - 1 ) + 4 {\hat{σ}}_{β}^{2} + 2 {\hat{σ}}_{ɛ}^{2} & - {\hat{μ}}^{2} ( {ⅇ}^{{\hat{λ}}^{2}} - 1 ) \\ - {\hat{μ}}^{2} ( {ⅇ}^{{\hat{λ}}^{2}} - 1 ) & {\hat{μ}}^{2} ( {ⅇ}^{{\hat{λ}}^{2}} - 1 ) + 2 {\hat{σ}}_{ɛ}^{2}\end{matrix} ) & ( 32 )\end{matrix}$$

6. The system according to claim 5 wherein said posterior probability calculating means calculates the posterior probability of expression of any gene in cell 1 and cell 2 for each pair (u, v) of the gene expression level data sent from said input device according to the following equation (33) (where f(u, ν|\hat{p}, \hat{θ}) is given by the following equation (34)) and the following equation (35) (where τ₁, τ₂ take either 1 or 0, which represents the presence or absence of true gene expression in each cell).
$$\begin{matrix}Pr ( {τ}_{1} = 1 \vert \hat{p} , \hat{θ} ) = \frac{{\hat{p}}_{10} {g}_{10} ( u , v \vert \hat{θ} ) + {\hat{p}}_{11} {g}_{11} ( u , v \vert \hat{θ} )}{f ( u , v \vert \hat{p} , \hat{θ} )} & ( 33 ) \\ f ( u , v \vert \hat{p} , \hat{θ} ) = {\sum }_{( j , k ) ∈ {\{ 0 , 1 \}}^{2}} {\hat{p}}_{jk} {g}_{jk} ( u , v \vert \hat{θ} ) & ( 34 )\end{matrix}$$
$$Pr ( {τ}_{2} = 1 \vert \hat{p} , \hat{θ} ) = \frac{{\hat{p}}_{01} {g}_{01} ( u , v \vert \hat{θ} ) + {\hat{p}}_{11} {g}_{11} ( u , v \vert \hat{θ} )}{f ( u , v \vert \hat{p} , \hat{θ} )} \qquad ( 35 )$$

7. The system according to claim 5 wherein said posterior probability calculating means calculates the posterior probability indicating an event of differential expression between cell 1 and cell 2 according to the following equation (36)
$$Pr ( {τ}_{1} \neq {τ}_{2} \vert \hat{p} , \hat{θ} ) = \frac{{\hat{p}}_{10} {g}_{10} ( u , v \vert \hat{θ} ) + {\hat{p}}_{01} {g}_{01} ( u , v \vert \hat{θ} )}{f ( u , v \vert \hat{p} , \hat{θ} )} \qquad ( 36 )$$
where τ¹, τ₂ take either 1 or 0, which represents the presence or absence of true gene expression in each cell.

8. The system according to claim 6 or 7 wherein said posterior probability calculating means judges whether calculations of posterior probabilities of gene expression have been made for all the pairs (u, v) of the gene expression level data, and when all the calculations have been completed, it ends the process, while when all the calculations have not been completed yet, it calculates the posterior probability related to the next gene, such that the calculated posterior probabilities of gene expression in each channel are sent to said output device, and
said output device displays the posterior probabilities of gene expression in each channel.

9. A gene expression state estimating method for estimating the probability of gene expression in each channel based on gene expression level data, comprising the steps of:
estimating distributed parameters of a mixed normal distribution shown in the following equation (37) using the gene expression level data and sending the estimated distributed parameters:
(1−ξ)φ(u−μ₀|σ₀²)+ξφ(u−μ₁|σ₁²) (37)
where φ(*|σ²) represents the density function of a one-dimensional normal distribution with average 0 and variance σ², (μ₀, σ₀²) and (μ₀, σ₁²) are average and variance parameters of first and second components, respectively, and t is the mixing ratio, with the assumption that μ₀<μ₁, σ₀²>0, σ₁²>0, 0<ξ<1 is satisfied,
estimating a mixing ratio parameter of the mixed normal distribution using the gene expression level data and the estimated distributed parameters, and sending the estimated mixing ratio parameter, and
calculating the posterior probability of the expression state of each gene in each channel using the gene expression level data, the estimated distributed parameters, and the estimated mixing ratio parameter, and sending the calculated posterior probability.

10. The method according to claim 9 wherein said step of estimating the distributed parameters further comprises a step of estimating the mixing ratio (ξ), average (μ₀, μ₁), and variance (σ₀², σ₁²) by applying the mixed normal distribution of two components to data on the sum of the amounts of expression of genes located in a region where the difference of gene expression levels X and Y of two channels is near 0.

11. The method according to claim 10 wherein when the median value of the absolute difference |νᵢ|(i=1, . . . , n) of the gene expression levels X and Y is c_{M}, the data on the sum of the amounts of expression of genes is shown by {uᵢ∥νᵢ|≦c_{M}, i=1, . . . ,n}.

12. The method according to claim 11 wherein the estimation is performed in said step of estimating the distributed parameters by the use of the estimated \hat{ξ}, \hat{μ}₀, \hat{σ}₀², \hat{μ}₁, \hat{σ}₁² according to the following equations (38), (39), (40), and (41) to estimate μ, σ_{ε}², σ_{β}², λ;
$$\begin{matrix}\hat{μ} = ( {\hat{μ}}_{1} - {\hat{μ}}_{0} ) / 2 & ( 38 ) \\ {\hat{σ}}_{ɛ}^{2} = \frac{1}{2 ‖ {N}_{0} ‖} {\sum }_{i ∈ {N}_{0}} {v}_{i}^{2} & ( 39 ) \\ {\hat{σ}}_{β}^{2} = \frac{1}{4} {\hat{σ}}_{0}^{2} - \frac{1}{2} {\hat{σ}}_{ɛ}^{2} & ( 40 ) \\ \hat{λ} = \sqrt{log ( 1 + \frac{{\hat{σ}}_{1}^{2} - {\hat{σ}}_{2}^{2}}{4 {\hat{μ}}^{2}} )} & ( 41 )\end{matrix}$$
where N₀ denotes an index set of data values that satisfies i ε {i|uᵢ<\hat{μ}₀} and ∥N₀∥ denotes the number of elements.

13. The method according to claim 12 wherein the estimation is performed in said step of estimating the mixing ratio parameter p=(p₀₀, p₁₀, p₀₁, p₁₁) (where p₀₀ denotes a mixing ratio when no gene is being expressed in both cells 1 and 2, p₁₁ denotes a mixing ratio when any gene is being expressed in both cells 1 and 2, p₁₀ denotes a mixing ratio when a gene is being expressed in cell 1 but not in cell 2, and g₀₁ represents a mixing ratio when a gene is being expressed in cell 2 but not in cell 1) using \hat{θ}=(\hat{μ}, \hat{λ}, \hat{σ}_{ε}², \hat{σ}_{β}²) sent from said step of estimating the distributed parameters by applying a two-variable mixed normal distribution of four components shown in the following equation (42) to the sent gene expression level data {(uᵢ, vᵢ) i=1, . . . ,n}
p₀₀g₀₀(u,ν|\hat{θ})+p₁₀g₁₀(u,ν|\hat{θ})+p₀₁g₀₁(u,ν|\hat{θ})+p₁₁g₁₁(u,ν|\hat{θ})=p₀₀φ(u|4\hat{σ}_{β}²+2\hat{σ}_{ε}²)φ(ν|2\hat{σ}_{ε}²)+p₁₀φ₂(u−\hat{μ},ν−\hat{μ}|Σ₁₀)+p₀₁φ₂(u−\hat{μ},ν+\hat{μ}|Σ₀₁)+p₁₁φ(u−2\hat{μ}|4\hat{μ}²(e^{λ}^{²}−1)+4\hat{σ}_{β}²+2\hat{σ}_{ε}²)φ(ν|2\hat{σ}_{ε}²) (42)
where the above equation satisfies the relationships shown in the following equations (43) and (44).
$$\begin{matrix}{\hat{\sum }}_{10} = ( \begin{matrix}{\hat{μ}}^{2} ( {ⅇ}^{{\hat{λ}}^{2}} - 1 ) + 4 {\hat{σ}}_{β}^{2} + 2 {\hat{σ}}_{ɛ}^{2} & {\hat{μ}}^{2} ( {ⅇ}^{{\hat{λ}}^{2}} - 1 ) \\ {\hat{μ}}^{2} ( {ⅇ}^{{\hat{λ}}^{2}} - 1 ) & {\hat{μ}}^{2} ( {ⅇ}^{{\hat{λ}}^{2}} - 1 ) + 2 {\hat{σ}}_{ɛ}^{2}\end{matrix} ) & ( 43 ) \\ {\hat{\sum }}_{01} = ( \begin{matrix}{\hat{μ}}^{2} ( {ⅇ}^{{\hat{λ}}^{2}} - 1 ) + 4 {\hat{σ}}_{β}^{2} + 2 {\hat{σ}}_{ɛ}^{2} & - {\hat{μ}}^{2} ( {ⅇ}^{{\hat{λ}}^{2}} - 1 ) \\ - {\hat{μ}}^{2} ( {ⅇ}^{{\hat{λ}}^{2}} - 1 ) & {\hat{μ}}^{2} ( {ⅇ}^{{\hat{λ}}^{2}} - 1 ) + 2 {\hat{σ}}_{ɛ}^{2}\end{matrix} ) & ( 44 )\end{matrix}$$

14. The method according to claim 13 wherein the calculation of the posterior probability of expression of any gene in cell 1 and cell 2 is made in said step of calculating the posterior probability for each pair (u, v) of the sent gene expression level data according to the following equation (45) (where f(u, ν|\hat{p}, \hat{θ}) is given by the following equation (46)) and the following equation (47) (where τ₁, τ₂ take either 1 or 0, which represents the presence or absence of true gene expression in each cell).
$$\begin{matrix}Pr ( {τ}_{1} = 1 \vert \hat{p} , \hat{θ} ) = \frac{{\hat{p}}_{10} {g}_{10} ( u , v \vert \hat{θ} ) + {\hat{p}}_{11} {g}_{11} ( u , v \vert \hat{θ} )}{f ( u , v \vert \hat{p} , \hat{θ} )} & ( 45 ) \\ f ( u , v \vert \hat{p} , \hat{θ} ) = {\sum }_{( j , k ) ∈ {\{ 0 , 1 \}}^{2}} {\hat{p}}_{jk} {g}_{jk} ( u , v \vert \hat{θ} ) & ( 46 ) \\ Pr ( {τ}_{2} = 1 \vert \hat{p} , \hat{θ} ) = \frac{{\hat{p}}_{01} {g}_{01} ( u , v \vert \hat{θ} ) + {\hat{p}}_{11} {g}_{11} ( u , v \vert \hat{θ} )}{f ( u , v \vert \hat{p} , \hat{θ} )} & ( 47 )\end{matrix}$$

15. The method according to claim 13 wherein the calculation of the posterior probability indicating an event of differential expression between cell 1 and cell 2 is made in said step of calculating the posterior probability according to the following equation (48)
$$Pr \text{(} {τ}_{1} \neq {τ}_{2} \vert \hat{p} , \hat{θ} \text{)} = \frac{{\hat{p}}_{10} {g}_{10} \text{(} u , ν \vert \hat{θ} \text{)} + {\hat{p}}_{01} {g}_{01} \text{(} u , ν \vert \hat{θ} \text{)}}{f \text{(} u , ν \vert \hat{p} , \hat{θ} \text{)}} \qquad ( 48 )$$
where τ₁, τ₂ take either 1 or 0, which represents the presence or absence of true gene expression in each cell.

16. The method according to claim 14 or 15 wherein it is judged in said step of calculating the posterior probability whether calculations of posterior probabilities of gene expression have been made for all the pairs (u, v) of the gene expression level data, and when all the calculations have been completed, the process is ended, while when all the calculations have not been completed yet, the posterior probability related to the next gene is calculated.

17. A gene expression state estimating program for estimating the probability of gene expression in each channel based on gene expression level data, the program instructing a computer to execute the steps of:
estimating distributed parameters of a mixed normal distribution shown in the following equation (49) using the gene expression level data and sending the estimated distributed parameters:
(1−ξ)φ(u−μ₀|σ₀²)+ξφ(u−μ₁|σ₁²) (49)
where φ(*|σ²) represents the density function of a one-dimensional normal distribution with average 0 and variance σ², (μ₀, σ₀²) and (μ₀, σ₁²) are average and variance parameters of first and second components, respectively, and t is the mixing ratio, with the assumption that μ₀<μ₁, σ₀²>0, σ₁²>0, 0<ξ<1 is satisfied,
estimating a mixing ratio parameter of the mixed normal distribution using the gene expression level data and the estimated distributed parameters, and sending the estimated mixing ratio parameter, and
calculating the posterior probability of the expression state of each gene in each channel using the gene expression level data, the estimated distributed parameters, and the estimated mixing ratio parameter, and sending the calculated posterior probability.

18. A computer-readable recording medium storing a gene expression state estimating program for estimating the probability of gene expression in each channel based on gene expression level data, the program instructing a computer to execute the steps of:
estimating distributed parameters of a mixed normal distribution shown in the following equation (50) using the gene expression level data and sending the estimated distributed parameters:
(1−ξ)φ(u−μ₀|σ₀²)+ξφ(u−μ₁|σ₁²) (50)
where φ(*|σ²) represents the density function of a one-dimensional normal distribution with average 0 and variance σ², (μ₀, σ₀²) and (μ₀, σ₁²) are average and variance parameters of first and second components, respectively, and t is the mixing ratio, with the assumption that μ₀<μ₁, σ₀²>0, σ₁²>0, 0<ξ<1 is satisfied,
estimating a mixing ratio parameter of the mixed normal distribution using the gene expression level data and the estimated distributed parameters, and sending the estimated mixing ratio parameter,
calculating the posterior probability of the expression state of each gene in each channel using the gene expression level data, the estimated distributed parameters, and the estimated mixing ratio parameter, and sending the calculated posterior probability, and
outputting the calculated posterior probability.
